(19) United States
(12) Patent Application Publication
(10) Pub. No.: US 2013/0018369 A1
(43) Pub. Date: Jan. 17, 2013

(54) SYSTEMS AND METHODS FOR TREATMENT IN PROXIMITY TO SENSITIVE TISSUE STRUCTURES

(71) Applicant: Teresa Ann MIHALIK, Encinitas, CA (US)

(72) Inventor: Teresa Ann MIHALIK, Encinitas, CA (US)

(73) Assignee: MEDTRONIC CRYOCATH LP, Toronto (CA)

(21) Appl. No.: 13/182,201

(22) Filed: Jul. 13, 2011

(51) Int. Cl. A61B 18/02; A61F 7/00

(52) U.S. Cl. 606/21; 607/96; 607/104; 607/113

(57) ABSTRACT

A method of treating a target tissue including positioning a treatment element of a medical device adjacent the target tissue; setting a first target temperature of the treatment element; operating the treatment element to reach the first target temperature; thermally treating the target tissue with the treatment element; monitoring a physiological parameter; setting a second target temperature of the treatment element based on the monitored physiological parameter; and operating the treatment element to reach the second target temperature.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method for modifying the temperature or power of a treatment element of a medical device based on physiological and anatomical information.

### BACKGROUND OF THE INVENTION

When treating particular regions of tissue, through thermal energy interaction or the like for example, it may be difficult to direct or control the depth and intensity of the heat transfer. The delivery of thermal energy or other therapeutic modality may not be necessarily contained to the exact region or depth desired for treatment, as the tissue may have varying therapy-conducive properties affected by the surrounding physiological environment. While thermal control or precision may be of more concern with certain treatment modalities, such as radiofrequency or microwave treatment procedures, it is often desirable to limit thermal treatment or exposure to just the tissue desired. Failure to do so may otherwise negatively and adversely affect surrounding tissue structures or organs that are sensitive and susceptible to undesired damage.

For example, when attempting to treat cardiac tissue, sensitive tissue structures abound that may react adversely to thermal applications. In particular, when thermally treating or ablating tissue in or about the heart, it is essential that critical physiological structures such as the phrenic nerve, sinoatrial node, esophagus, and the like are not inadvertently destroyed through such ablation therapy. The phrenic nerve is made up mostly of motor nerve fibers that produce contractions of the diaphragm and thus affect breathing and respiration patterns and conditions. In addition, the phrenic nerve provides sensory innervation for many components of the mediastinum and pleura, as well as the upper abdomen, especially the liver, and the gall bladder.

The phrenic nerve is generally referred to in two segments: the right and left phrenic nerves. Both phrenic nerves run from C3, C4 and C5 vertebrae along the anterior scalene muscle deep to the carotid sheath. The right phrenic nerve passes over the brachlocephalic artery, posterior to the subclavian vein, and then crosses the root of the right lung anteriorly and then leaves the thorax by passing through the vena cava hiatus opening in the diaphragm at the level of T8. The right phrenic nerve passes over the right atrium. The left phrenic nerve passes over the pericardium of the left ventricle and pierces the diaphragm separately.

Referring to FIGS. 1-3, the close proximity of the phrenic nerve segments to the right atrium and left ventricle is illustrated. These cardiac regions may be the location or origin of heart arrhythmias or other physiological maladies and thus targeted for tissue ablation in order to remove or otherwise remedy the abnormal electrophysiological occurrence. In thermally treating or ablating select cardiac regions, the phrenic nerve may be at risk of being similarly, although unintentionally, ablated. This could severely impact the normal respiratory functioning of the patient. The risk of such unintentional and undesirable destruction or application of thermal energy to this and other cursory structures compels a desire to precisely determine the location and position of the ablation device relative to the desired tissue to be treated, and to modulate or temper such treatment given the proximity to sensitive structures.

In particular, it is advantageous to systems and methods of use thereof that modulate or temper treatment regimens based at least in part on the presence of sensitive tissue structures in the general treatment vicinity.

### SUMMARY OF THE INVENTION

The present invention advantageously provides a method of treating a target tissue, including positioning a treatment element of a medical device adjacent the target tissue; setting a first target temperature of the treatment element; operating the treatment element to reach the first target temperature; thermally treating the target tissue with the treatment element; monitoring a physiological parameter; setting a second target temperature of the treatment element based on the monitored physiological parameter; and operating the treatment element to reach the second target temperature. The second target temperature may be greater than or less than the first target temperature depending upon changes in the monitored physiological parameter. The method may include assessing a distance between the treatment element and an anatomical structure spaced from the target tissue; and the first targeting temperature setting may be based on at least part on the assessed distance. The anatomical structure may include a phrenic nerve and/or esophagus. Operating the treatment element may include circulating a cryogenic fluid through the treatment element; modifying an internal pressure within the treatment element; and/or circulating an insulating fluid within a portion of the treatment element. Thermally treating the target tissue may include inducing a reversible physiological response and/or ablating the target tissue. Monitoring a physiological parameter may include monitoring electrical activity of the target tissue with the medical device, where the physiological parameter may include at least one of breathing rate, esophageal temperature, body temperature, diaphragm impedance, phrenic nerve activity and/or electrical activity in proximity to at least one of a bundle of his or atrioventricular node. The method may also include monitoring a temperature of the treatment element, and modulating operation of the treatment element to substantially maintain the first target temperature.

A method for treating a patient is provided, including measuring a distance between a thermal treatment element of a medical device and an anatomical structure of the patient; setting a target temperature threshold based on the measured distance; operating the treatment element within the target temperature threshold to thermally treat a target tissue different from the anatomical structure; monitoring a physiological parameter during the operation of the treatment element; and modifying the target temperature threshold based at least in part on the monitored physiological parameter. The monitored physiological parameter may include at least one of breathing rate, esophageal temperature, body temperature, diaphragm impedance, or phrenic nerve activity. The anatomical feature may include at least one of the phrenic nerve, bundle of His, or the atrioventricular node. Thermally treating the target tissue may include cryogenically ablating the target tissue, and operating the treatment element may include circulating a cryogenic fluid toward the treatment element, the treatment element having a first balloon surrounding a second balloon and an interstitial region enclosing a volume defined therebetween, and modifying the volume within the interstitial region.

A method for thermally treating a patient is disclosed, including measuring a distance between a thermal treatment element and an anatomical feature; defining a first operating temperature range based on the measured distance; delivering a cryogenic fluid to the treatment element to reach a temperature within the first operating temperature range; conducting thermal energy between a target tissue and the treatment element; monitoring a physiological parameter of the patient; defining a second operating temperature range based at least in part on the monitored physiological parameter; and modifying delivery of the cryogenic fluid to the treatment element to reach a temperature within the second operating temperature range.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an anterior illustration of a thoracic region and related anatomy;

FIG. 2 is an illustration of a human heart and related anatomy;

FIG. 3 is an additional illustration of a human heart and related anatomy;

FIG. 4 is an illustration of a medical device system constructed in accordance with the principles of the present invention;

FIG. 5a is a front view of a positioning system constructed in accordance with the principles of the present invention;

FIG. 5b is a back view of the positioning system shown in FIG. 5a;

FIG. 6 is a flow-chart of a method in accordance with the principles of the present invention; and

FIG. 7 is a graph showing the change in temperature over time of a treatment element of a medical device based on monitored physiological conditions.

### DETAILED DESCRIPTION OF THE INVENTION

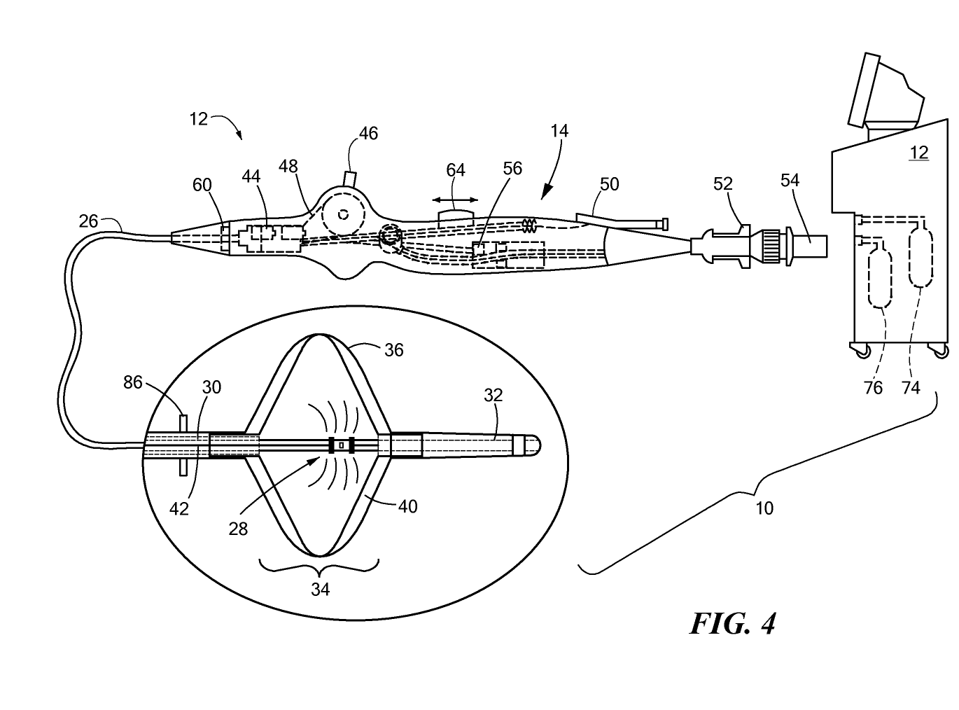

The present invention advantageously provides a method and system for identifying the position of a treatment element of a medical device with respect to a particular anatomical structure or physiological and setting and/or modifying the temperature of the treatment element based on its position. Referring now to the drawing figures in which like reference designations refer to like elements, an embodiment of a medical system constructed in accordance with principles of the present invention is shown in FIG. 4 and generally designated as “10.” The system generally includes a cooling unit or controller 12 coupled to a medical device 14 through an umbilical system or other connectors. The medical device 14 may be a medical probe, a catheter, a balloon-catheter, as well as other devices deliverable or otherwise positionable through the vasculature and/or proximate to a tissue region for treatment. In particular, the medical device 14 may include a device operable to thermally treat a selected tissue site, including blood vessels and adjacent structures, cardiac or renal tissue. The medical system 10 may also include one or more sensors to monitor the operating parameters throughout the system, including for example, pressure, temperature, flow rates, volume, or the like in the controller 12, the umbilical system, and/or the medical device 14.

The medical device 14 may include an elongate body 26 passable through a patient's vasculature. The elongate body 26 may define a proximal portion and a distal portion, and may further include one or more lumens may disposed within the elongate body 26 thereby providing mechanical, electrical, and/or fluid communication between the proximal and distal portions of the elongate body 26. For example, the elongate body 26 may include an injection lumen 28 and an exhaust lumen 30 defining a fluid flow path therethrough. In addition, the elongate body 26 may include a guide wire lumen 32 movably disposed within and/or extending along at least a portion of the length of the elongate body 26 for over-the-wire applications. The guide wire lumen 32 may define a proximal end and a distal end, and the guide wire lumen 32 may be movably disposed within the elongate body 26 such that the distal end of the guide wire lumen 32 extends beyond and out of the distal portion of the elongate body 26.

The medical device 14 may include one or more treatment regions for energetic or other therapeutic interaction between the medical device 14 and a treatment site. The treatment regions may deliver, for example, radiofrequency energy, cryogenic therapy, or the like. For example, the device 14 may include a first treatment region 34 having a thermal treatment element, such as an expandable membrane or balloon and/or one or more electrodes or other thermally-transmissive components, or sensors, at least partially disposed on the elongate catheter body 26. In a particular example, the first treatment region 34 may include a first expandable/inflatable element or balloon 36 defining a proximal end coupled to the distal portion of the elongate body 26 of the medical device 14, while further defining a distal end coupled to the distal end of the guide wire lumen 32. As such, due to the movable nature of the guide wire lumen 32 about the elongate body 26, any axial and/or longitudinal movement of the guide wire lumen 32 may act to tension or loosen the first expandable element 36, i.e., extend or retract the expandable element 36 from a lengthened state to a shortened state during an inflation or deflation thereof. In addition, the first expandable element 36 may define and the desired operating temperature, or temperature any of a myriad of shapes, and may further include one or more material layers providing for puncture resistance, radiopacity, or the like. The first expandable element 36 may be in communication with the fluid injection and exhaust lumens of the medical device 14 as described above.

The medical device 14 may further include a second expandable/inflatable element or balloon 38 contained within or otherwise encompassed by the first expandable element 36 such that an interstitial region, envelope or space 40 is defined therebetween. The second expandable element 38 may be in communication with the fluid injection and exhaust lumens of the medical device 14 as described above, i.e., a fluid flow path may provide an inflation fluid or coolant, such as a cryogenic fluid or the like, to the interior of the second expandable element 38. Further, the interstitial region 40 may be in fluid communication with an interstitial lumen 42 providing a fluid flow path or avenue separate and independent from a fluid flow path delivering fluid or otherwise in communication with an interior of the second expandable element 38. The second pathway provides an alternate exhaust route for fluid that may leak from the interior of the second expandable element 38 into the interstitial region 40 or fluid entering the medical device 14 from the exterior. In particular, the isolation of the interstitial lumen 42 from the interior of the second expandable element 38 provides an alternate route for fluid to circulate in the case of a rupture or leak of either the first or second expandable elements, as well as allowing for the injection or circulation of fluids within the interstitial region 40 independently of fluids directed towards the second expandable element 38. Towards that end, the interstitial region 40 may be in fluid communication with a fluid source, a vacuum source, or the like separate from a fluid source, vacuum source or otherwise in fluid communication with the interior of the second expandable element 38. Alternatively, the interstitial lumen 42 may be joined to or otherwise in fluid communication with the injection lumen 28 and the interior of the second expandable element 38 to provide a single exhaust or vacuum source for the medical device 14.

Continuing to refer to FIG. 4, the size of the interstitial region 40 and/or relative spacing between the first expandable element 36 and the second expandable element 38 may be selectively and independently adjustable based on the desired treatment to be performed, and/or based on the desired operating temperature, or operating temperature range of the treatment element. For example, if a cryogenic ablative treatment is to be performed, the spacing between the first expandable element 36 and the second expandable element 38 may be reduced, for example, approximately 0 mm, such that thermal resistance between the second expandable member 38 and the first expandable member 36 is minimized. Alternatively, the spacing between the first expandable element 36 and the second expandable element 38 may be increase to a distance of approximately 2 mm for non-ablative treatments. This adjustment in spacing may be accomplished, for example, by evacuating the air space in the interstitial region 40 to a desired volume. Alternatively, a fluid may be circulated through the interstitial lumen 42 towards the interstitial region 40 to provide the spacing between the first expandable element 36 and the second expandable element 38. For example, the first expandable element 36 may be spaced from the second expandable element 38 by the volume of the interstitial region fluid. In another configuration, the pressure of the interstitial region 40 may also be adjusted, such that the first expandable member 36 is a spaced a predetermined distance away from the second expandable member 38. The regulation of the volume or fluid, spacing, and/or pressure within the interstitial region 40 may be achieved by the controller 12 or by a second controller (not shown) either disposed within the controller 12 or which may be a remote controller in communication with the controller 12.

Should the user or physician desire to maintain the temperature of the first expandable element 36 at a higher temperature than the target cryogenic ablation temperature, for example for mapping of the target tissue region and/or surrounding anatomical or physiological structures, the size of the interstitial region 40 may be increased. In particular, it is desirable for mapping tissue that the temperature of first expandable member 36 be sufficiently cold to temporarily paralyze, or impart a reversible electrophysiological effect on the tissue, but not to ablate it. In such conditions, and at such locations within the body, it is desirable to selectively increase the spacing between the first expandable element 36 and the second expandable element 38 such that temperature of the first expandable element 36 is higher than the second expandable member 38. For example, if a particular tissue is to be mapped and cryogenic fluid is circulated within the second expandable element 38, then it is desirable to create sufficient distance between the cryogenic fluid and the target tissue, such that when the first expandable element 36 contacts the target tissue it is sufficiently warm to avoid ablating the tissue.

As an alternative to the expandable member spacing methods discussed above, a biocompatible insulating fluid, may be circulated through the interstitial lumen 42 toward the interstitial region 40. The insulating fluid may operate to transfer heat to the cryogenic fluid circulating within the second expandable member 38 such that the first expandable member 36 is warmer than the second expandable member 38. In a particular configuration, tank 76 is provided within controller 12 and houses the insulating fluid. The interstitial lumen 42 may be selectively switchable from being in fluid communication with the vacuum pump and tank 76 such that the controller can regulate the size and contents of the interstitial region 40 depending on the desired procedure.

As another alternative, the fluid flow rate of refrigerant delivered to the second expandable member 38 may be regulated to increase the power. For example, depending on the size of the first expandable member 36 and the second expandable member 38, the fluid flow rate of refrigerant into the second expandable member 38 may be adjusted based on the desired temperature and the size of the second expandable member 38. In a particular example in which the second expandable member 38 is approximately 28 mm in diameter and the flow rate of fluid into the second expandable member is approximately 6.2 liters/minute, if the diameter of the second expandable member 38 is reduced to approximately 23 mm and the flow rate remains unchanged, then the temperature of the 23 mm expandable member may be approximately 5-10 degrees Celsius colder than the temperature of the 28 mm expandable member owing to the increase in power per surface area. Similarly, if the flow rate of refrigerant into the 28 mm expandable member is adjusted, for example, by 0.5 liter/minute increments, the temperature of the second expandable member 38 may be incrementally increased or decreased depending on the desired temperature.

Continuing to refer to FIG. 4, the medical device 14 may include a handle 44 coupled to the proximal portion of the elongate body 26, where the handle 44 may include an element such as a lever or knob 46 for manipulating the catheter body and/or additional components of the medical device 14. For example, a pull wire 48 with a proximal end and a distal end may have its distal end anchored to the elongate body 26 at or near the distal end. The proximal end of the pull wire 48 may be anchored to an element such as a cam in communication with and responsive to the lever 46. The handle 44 can further include circuitry for identification and/or use in controlling of the medical device 14 or another component of the system.

Additionally, the handle may be provided with a fitting 50 for receiving a guidewire that may be passed into the guidewire lumen 32. The handle 44 may also include connectors that are matable directly to a fluid supply/exhaust and control unit or indirectly by way of one or more umbilicals. For example, the handle may be provided with a first connector 52 that is matable with the co-axial fluid umbilical 18 and a second connector 54 that is matable with the electrical umbilical 20. The handle 44 may further include blood detection circuitry 56 in fluid and/or optical communication with the injection, exhaust and/or interstitial lumens. The handle 44 may also include a pressure relief valve 58 in fluid communication with the injection, exhaust and/or interstitial lumens to automatically open under a predetermined threshold value in the event that value is exceeded.

The medical device 14 may further include one or more temperature, pressure, and/or flow sensors proximate or otherwise in communication with the treatment region(s) positioned for monitoring, recording and/or conveying measurements of conditions within the medical device 14 or the ambient environment at the distal portion of the medical device 14. The sensor(s) may be in communication with the controller 12 for initiating or triggering one or more alerts or therapeutic delivery modifications during operation of the medical device 14. For example, the handle may include one or more pressure sensors 60 to monitor the fluid pressure within the lumens and/or treatment region of the medical device 14. The handle may also include one or more fluid flow rate sensors 62 to measure or record the fluid flow rate and/or an amount of fluid introduced into the lumens or treatment regions of the medical device 14 for any given time period. It is also contemplated that such sensors may be included or otherwise housed within the controller 12 and its components, as described below.

Continuing to refer to FIG. 4, the medical device 14 may include an actuator element 64 that is movably coupled to the proximal portion of the elongate body 26 and/or the handle 44. The actuator element 64 may further be coupled to the proximal portion of the guide wire lumen 32 such that manipulating the actuator element 64 in a longitudinal direction causes the guide wire lumen 32 to slide towards either of the proximal or distal portions of the elongate body 26. As a portion of either and/or both the first and second expandable elements 36, 38 may be coupled to the guide wire lumen 32, manipulation of the actuator element 64 may further cause the expandable element(s) to be tensioned or loosened, depending on the direction of movement of the actuator element 64, and thus, the guide wire lumen 32. Accordingly, the actuator element 64 may be used to provide tension on the expandable element(s) 36, 38 during a particular duration of use of the medical device 14, such as during a deflation sequence, for example. The actuator element 64 may include a thumb-slide, a push-button, a rotating lever, or other mechanical structure for providing a movable coupling to the elongate body 26, the handle 44, and/or the guide wire lumen 32. Moreover, the actuator element 64 may be movably coupled to the handle 44 such that the actuator element 64 is movable into individual, distinct positions, and is able to be releasably secured in any one of the distinct positions.

In an exemplary system, a fluid supply or reservoir 66 including a coolant, cryogenic refrigerant, or the like, an exhaust or scavenging system (not shown) for recovering or venting expended fluid for re-use or disposal, as well as various control mechanisms for the medical system may be housed in the controller 12. In addition to providing an exhaust function for the catheter fluid supply, the controller 12 may also include pumps, valves, controllers or the like to recover and/or re-circulate fluid delivered to the handle 44, the elongate body 26, and treatment region 34 of the medical device 14. A vacuum pump in the controller 12 may create a low-pressure environment in one or more conduits within the medical device 14 so that fluid is drawn into the conduit(s) of the elongate body 26, away from the treatment region 34, and towards the proximal end of the elongate body 26. The controller 12 may include one or more controllers, processors, and/or software modules containing instructions or algorithms to provide for the automated operation and performance of the features, sequences, or procedures described herein.

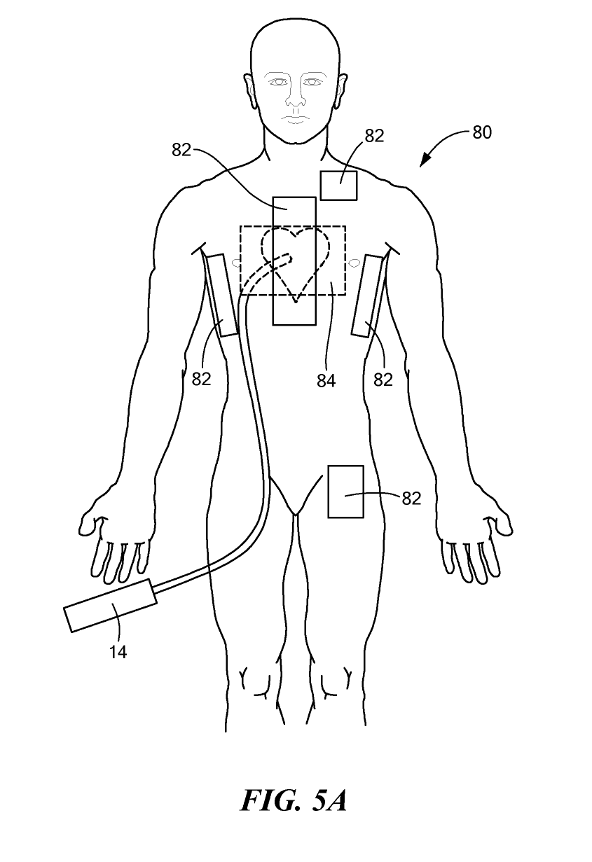

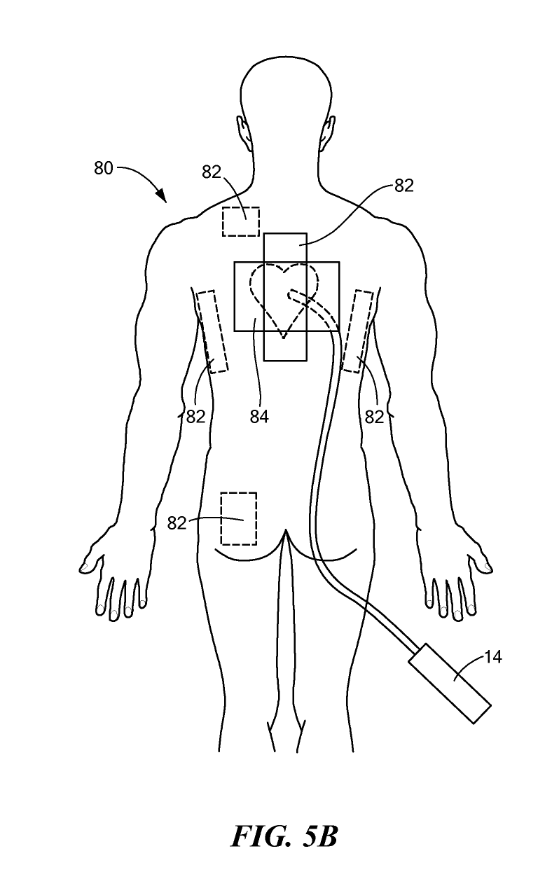

Now referring to FIGS. 5a and 5b, the medical system 10 may further include a medical device location and positioning system 80. The positioning system 80 may include one or more surface electrodes 82 positionable about the exterior of the patient (such as on the skin, for example) that include impedance measurement capabilities or other features allowing the detection, receipt, and/or transmission of an electrical signal. Each surface electrode 82 may have an adhesive surface that is removably affixable to the skin, or each surface electrode 82 may be implanted under the skin. Optionally, conductive gel (not shown) may be applied to the skin subjacent to the surface electrode 82 to increase the conductivity or adhesiveness between surface electrodes 82 and the body of the patient.

In an exemplary embodiment, three pairs of surface electrodes 82 are adhered to the skin to provide three-dimensional position information in x, y, and z planes. For example, as shown in FIGS. 5a and 5b, surface electrodes 82 include surface electrodes 82 adhered to the right and left sides of the chest of the patient; surface electrodes 82 adhered to portions of the neck and thigh respectively; and surface electrodes 82 adhered to the chest and back respectively.

Additional surface electrodes 82 may be adhered to the skin of the patient as desired in any number of desired locations to provide additional positional information, precision, and/or accuracy, as described more below. The surface electrodes 82 may be connected to a power supply, such as a generator, a display, and/or other signal processing components (such as those disclosed in U.S. Pat. Nos. 5,697,377 and 5,983,126, the entirety of each of which is incorporated herein by reference) to process and display information regarding or relating to the electrical signals sensed by the surface electrodes 82.

Continuing to refer to FIG. 5b, in addition to surface electrodes 82, at least one reference electrode 84 may be adhered to the skin of the patient in a similar fashion to that of the surface electrodes 82, or alternatively be disposed within the body of the patient, for example, at a location proximate or within the heart. For example, as shown in FIG. 4b reference electrode 84 may be adhered to the back of the patient on opposite sides of surface electrode 82. Reference electrode 84 may be grounded such that they are operable to receive RF energy during an RF energy treatment. Alternatively, reference electrode 84 may include a grounded, non-adhesive plate positionable beneath the patient. Additionally, any or all of the surface electrodes 82 may be selectively operable as ground electrodes during, for example, delivery of RF energy in a unipolar mode, as discussed in more detail below.

Localization and triangulation of the treatment element of the medical device 14 may be obtained by measuring and recording the electric potential or impedance activity between two or more of the surface electrodes 82 and between one or more of the surface electrodes 82 and at least one electrode 86 coupled to the medical device 14 proximate the treatment element. For example, an electric potential, which may be orthogonal, may be applied across surface electrodes 82 in sequence or simultaneously and sensed and measured by electrodes 86. From this measurement of electric potential or impedance, the position of the medical device 14 in x, y, and z planes may be extrapolated based on the known or calculated interelectrode distances between the surface electrodes 82. Any of the electrodes 86 may be further selectively operable to extrapolate the position of a portion of the medical device 12 for increased accuracy.

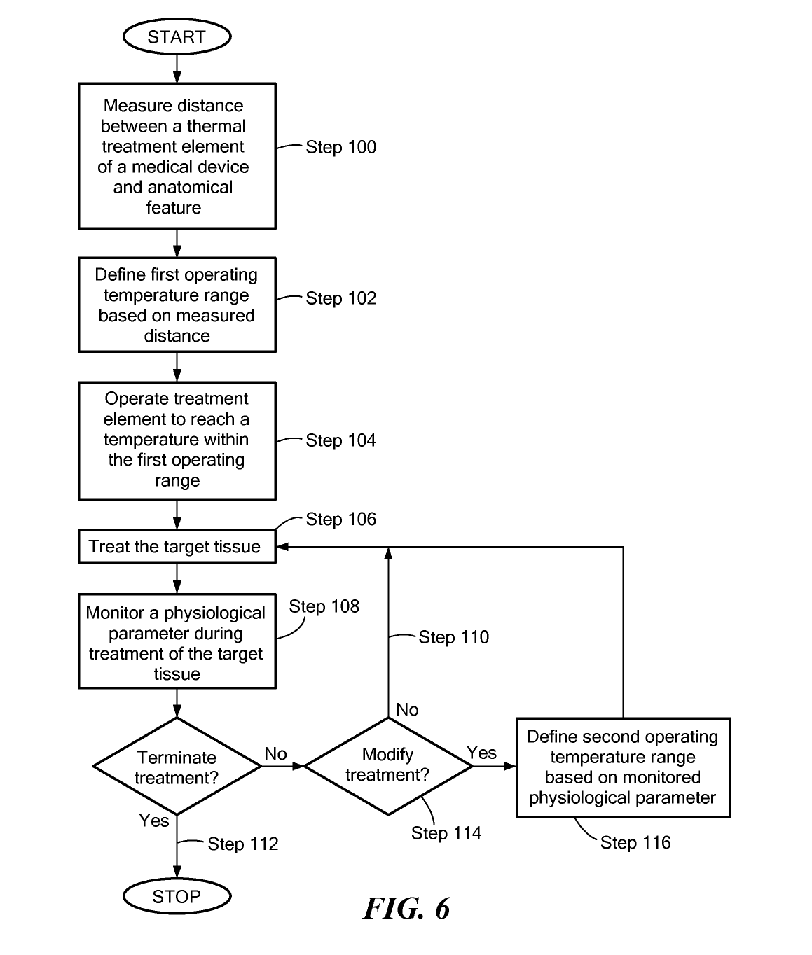

Now referring to FIG. 6 where an exemplary method of modifying the temperature of a treatment element of a medical device 14 based on the location and position of the treatment element with respect to particular anatomical or physiological structures in proximity to the tissue to be treated. A medical device 14 having a thermal energy transfer treatment element, for example, cryogenic, radiofrequency, microwave, electroporation, laser, etc., may be positioned within the patient, either through an incision proximate the heart, for example, through a sub-xyphoid surgical incision, minimally invasively through the vasculature, or other known body entry methods. The treatment element may be navigated towards the target treatment region, which may include guiding a catheter through the vasculature towards the target tissue region.

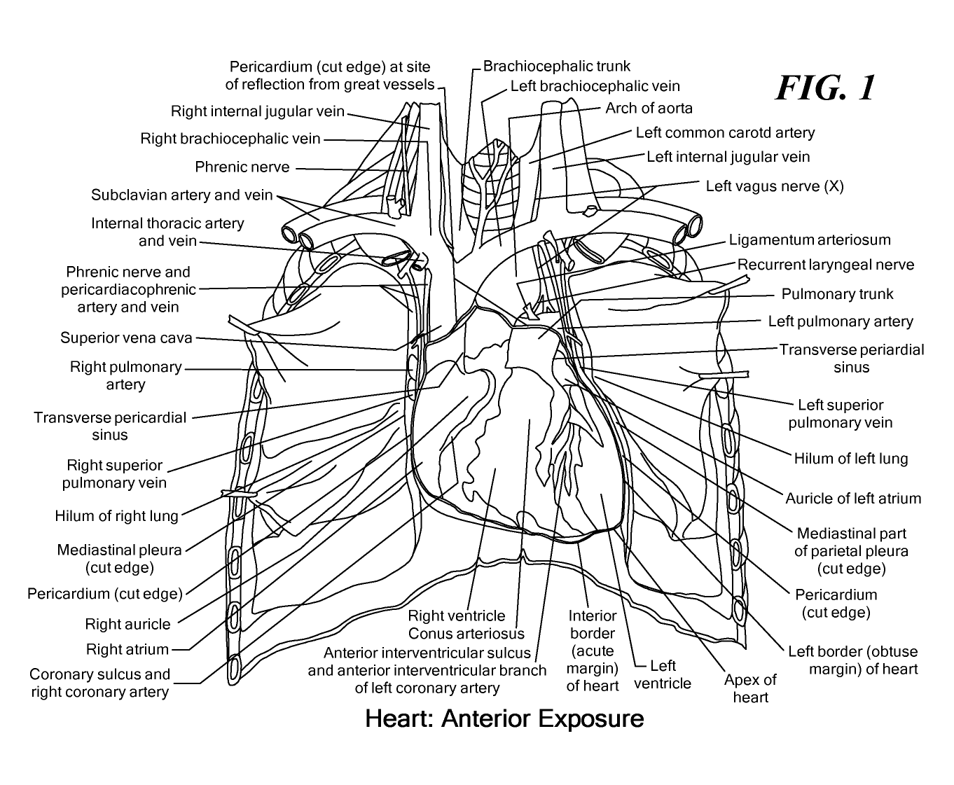

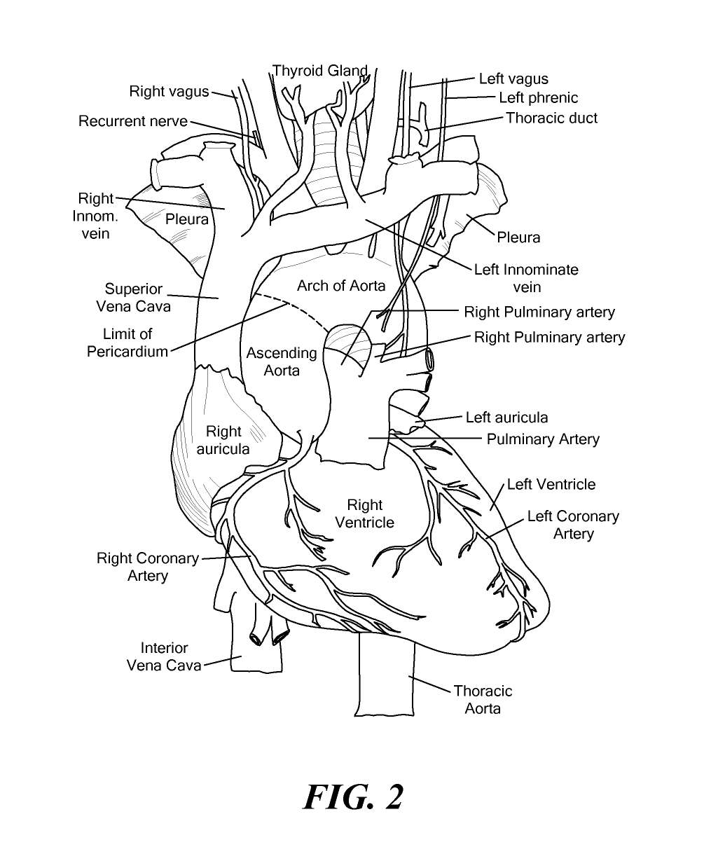

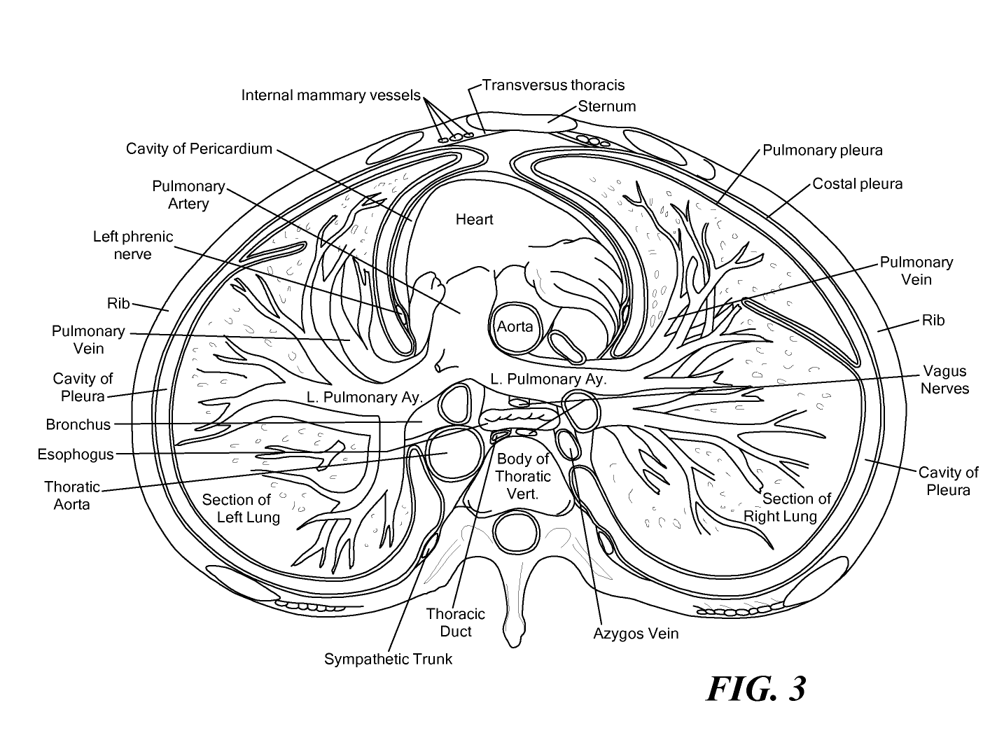

The position and location of the treatment element may be determined by the positioning system 80 discussed above and a distance between the treatment element and a particular anatomical feature, structure, or tissue may be measured. (Step 100). For example, the distance, in microns, millimeters, or centimeters, between any portion of the treatment element, for example an electrode, and an anatomical structure or feature. Examples of anatomical structures that may be proximate the treatment element include those structures shown in FIGS. 1-3, and in particular, the bundle of HIS, the atrioventricular node, the phrenic nerve, esophagus, or any structure of which inadvertent ablation may cause serious harm or death to a patient. Using imaging systems, such as MRI, CT, fluoroscopy, and/or 3D echocardiography, the anatomical information identified above, and physiological information, such as electrical impulses, blood flow, and oxygen concentration, or glucose concentration, may be visually and/or graphically identified on a display monitor along with a visual and/or graphical representation of the treatment element. A ruler, grid, or other measurement device may be visually displayed on the display such that the distance between the treatment element and a particular anatomical feature may be determined by a processor and/or visually by the surgeon.

Based on the measured distance between the treatment element and the particular anatomical structure spaced from the tissue to be treated, a target temperature, or operating temperature range, of the treatment element may be determined or set by the controller 12 (Step 102). For example, based on the measured distance between the treatment element and the sensitive tissue structure to be avoided, the surgeon or the controller 12 may choose a target temperature, or temperature range, for the treatment element based on tabulated clinical determinations or prior patient data. For example, software in the controller 12 may determine that based on a measured distance “x,” for example, 2-3 mm from the phrenic nerve, the temperature for the treatment element may be “y,” for example, approximate −50 degrees Celsius. The temperature “y” based on the distance “x” may be determined by an algorithm, may be variable depending on the particular patient's history, may be static, or may updatable based on additional information.

The treatment element, which may operate by utilizing cryogenic, RF, microwave, laser, or any other modality, may be operated to achieve the target temperature, or temperature range, based on the measured distance (Step 104). For example, in a particular configuration, if the surgeon or controller 12 determines that exchanging cryogenic energy with the target tissue region is appropriate, and ablating the target tissue is desired, the spacing between the first expandable member 36 and the second expandable member 38 may be reduced to minimize thermal resistance, and to lower the temperature at the tissue-first expandable member 36 interface. For example, the vacuum pump may suction a volume or air from the interstitial space 40 such that the second expandable member 38 is mechanically placed in contact with the first expandable member 36. Alternatively, the controller 12, or another control system in communication with the controller 12, may lower the pressure within the interstitial space 40 to provide for colder temperatures. Also, the flow rate of cryogenic fluid into the second expandable member 38 may be increased to provide for a colder temperature inside the second expandable element 38.

Cryogenic fluid may be circulated within the second expandable member 38 for a desired treatment time and the target tissue may be ablated (Step 106).

If the surgeon and/or the controller 12 determine that the target tissue should not be ablated, and the temperature of the treatment element should be increased, the size of the interstitial region 40 may be increased by circulating a fluid through the interstitial lumen 42 to fill the gap between the expandable members to increase the target temperature of the treatment element. The fluid may be saline, or a biocompatible insulating fluid to further increase the thermal resistance. Alternatively, the pressure within the second expandable element 38 may be raised such that the cryogenic fluid boils at a higher temperature, and thus the temperature within the second expandable member 38 is raised. Also, the flow rate of cryogenic fluid into the second expandable member 38 may be reduced such that temperature within the second expandable member 38 is higher. Cryogenic fluid may be circulated within the second expandable member 38 for a desired mapping time and the target tissue may be mapped.

In an exemplary mapping procedure, the treatment element is positioned proximate the target tissue. The temperature of the treatment element may be cold enough, for example, −30 degrees Celsius, reversibly cool the target tissue sufficient to temporarily impede electrical signal conduction, without permanently destroying or ablating the tissue. For example, the target tissue may be temporarily paralyzed to determine if a designated site is an arrhythmia locus.

During treatment of the target tissue, a physiological condition or parameter maybe measured and monitored (Step 108). For example, the patient's heart rate, breathing rate, blood pressure, blood flow rate, VO₂ MAX, heart electrical activity, diaphragm impedance, esophageal temperature, phrenic nerve activity, among other parameters may be monitored during treatment to determine whether treatment is adversely affecting the sensitive tissue structures in proximity to the treatment site (Step 108). In a specific example, a mapping wire (not shown) may be provided with the medical device 14 in electrical communication with the controller 12 and with the treatment region of the medical device 14. The mapping wire may be operable to provide real-time monitoring of pulmonary vein potentials, which may be used to determine whether to increase of decrease the temperature of the treatment element. For example, if all but one pulmonary vein potential is eliminated, the anatomy of the target tissue region may be too complex for the shape of the second expandable member 38, and a higher power may isolate the tissue and eliminate the remaining potential. If all potentials are all still present during treatment, then the target tissue region is sufficiently thick, such that the ablation is not transmural. The mapping wire mapping wire thus, in response to the measured potentials, may direct the controller 12 to lower the temperature of the treatment element by the methods discussed above.

Based on the monitored physiological parameters, the surgeon and/or the controller 12 may decide to continue treatment at the same temperature (Step 110) or terminate the treatment (Step 112) or modify the treatment (Step 114). If treatment is continued and modified, a second temperature of the treatment element may be set or determined based on the monitored physiological condition or parameter (Step 116). For example, a significant change in breathing rate during a procedure may indicate that the delivered treatment is affecting the phrenic nerve, and a determination to modify or terminate treatment results either automatically within the console or by alerting an operator to take action. Disruptions in the normal electrical and/or contractile rhythm of the heart result may reflect inadvertent impact of the delivered treatment on the SA node, and thus, treatment may be modified or terminated. If the monitored physiological condition or parameter remains in an acceptable range, the temperature of the treatment element may be reduced, i.e. ablation power is increased, to ablate the tissue region.

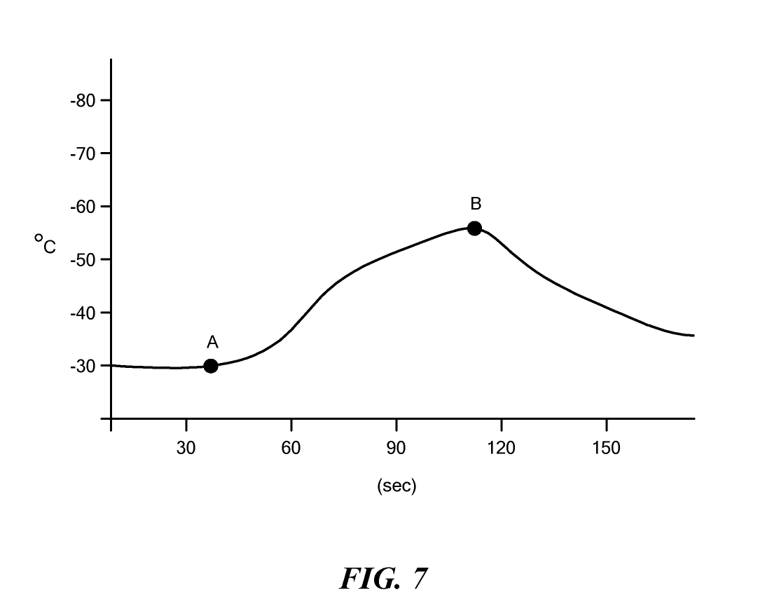

As an alternative to the treatment method discussed above, the treatment element may be operated at a non-ablative temperature before any measurement of distance in Step 102. For example, as shown in FIG. 7, the treatment element may be initially set at a temperature, for example, approximately −25 to −30 degrees Celsius for mapping. The anatomical and physiological parameters may be monitored by the methods discussed above during the mapping procedure, such that the temperature of the treatment element may be adjusted incrementally. In particular, as shown in FIG. 7, points “A” and “B” on the curve illustrated exemplary interrogations points, wherein the user and/or controller assesses the monitored parameter and decides whether to modify the treatment temperature. For example, the temperature of the treatment element may gradually be adjusted to a temperature sufficiently cold to permanently ablate tissue, for example, approximately −50 degrees Celsius, if the controller 12 and/or physician determines it is safe to do so based on monitored physiological and anatomical parameters. If negative side effects are monitored during an ablative treatment, the temperature of the treatment element may be raised to a level between a mapping temperature and an ablative temperature to derive an effect from the treatment.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of treating a target tissue, comprising:
positioning a treatment element of a medical device adjacent the target tissue;
setting a first target temperature of the treatment element;
operating the treatment element to reach the first target temperature;
thermally treating the target tissue with the treatment element;
monitoring a physiological parameter;
setting a second target temperature of the treatment element based on the monitored physiological parameter; and
operating the treatment element to reach the second target temperature.

2. The method of claim 1, further comprising assessing a distance between the treatment element and an anatomical structure spaced from the target tissue; and wherein the first targeting temperature setting is based on at least part on the assessed distance.

3. The method of claim 2, wherein the anatomical structure is at least one of a phrenic nerve, atrioventricular node, or his bundle.

4. The method of claim 2, wherein the anatomical structure is the esophagus.

5. The method of claim 1, wherein operating the treatment element includes circulating a cryogenic fluid through the treatment element.

6. The method of claim 5, wherein operating the treatment element includes modifying an internal pressure within the treatment element.

7. The method of claim 5, wherein operating the treatment element includes circulating an insulating fluid within a portion of the treatment element.

8. The method of claim 1, wherein thermally treating the target tissue includes inducing a reversible physiological response.

9. The method of claim 1, wherein thermally treating the target tissue includes ablating the target tissue.

10. The method of claim 1, wherein monitoring a physiological parameter includes monitoring electrical activity of the target tissue with the medical device.

11. The method of claim 1, wherein the physiological parameter includes at least one of breathing rate, esophageal temperature, body temperature, diaphragm impedance, or phrenic nerve activity.

12. The method of claim 1, further comprising:
monitoring a temperature of the treatment element, and
modulating operation of the treatment element to substantially maintain the first target temperature.

13. The method of claim 1, wherein the second target temperature is less than the first target temperature.

14. The method of claim 1, wherein the second target temperature is greater than the first target temperature.

15. A method for treating a patient, comprising:
measuring a distance between a thermal treatment element of a medical device and an anatomical structure of the patient;
setting a target temperature threshold based on the measured distance;
operating the treatment element within the target temperature threshold to thermally treat a target tissue different from the anatomical structure;
monitoring a physiological parameter during the operation of the treatment element; and
modifying the target temperature threshold based at least in part on the monitored physiological parameter.

16. The method of claim 15 wherein the monitored physiological parameter is at least one of breathing rate, esophageal temperature, body temperature, diaphragm impedance, or phrenic nerve activity.

17. The method of claim 15, wherein the monitored physiological parameter includes electrical activity in proximity to at least one of a His bundle or atrioventricular node.

18. The method of claim 15, wherein the anatomical feature is at least one of the phrenic nerve, bundle of His, or the atrioventricular node.

19. The method of claim 15, wherein thermally treating the target tissue includes cryogenically ablating the target tissue.

20. The method of claim 19, wherein operating the treatment element includes circulating a cryogenic fluid toward the treatment element, the treatment element having a first balloon surrounding a second balloon and an interstitial region enclosing a volume defined therebetween.

21. The method of claim 20, wherein operating the treatment element further includes modifying the volume within the interstitial region.

22. A method for thermally treating a patient, comprising:
measuring a distance between a thermal treatment element and an anatomical feature;
defining a first operating temperature range based on the measured distance;
delivering a cryogenic fluid to the treatment element to reach a temperature within the first operating temperature range;
conducting thermal energy between a target tissue and the treatment element;
monitoring a physiological parameter of the patient;
defining a second operating temperature range based at least in part on the monitored physiological parameter; and
modifying delivery of the cryogenic fluid to the treatment element to reach a temperature within the second operating temperature range.
